(19) United States
(12) Patent Application Publication
(10) Pub. No.: US 2017/0154158 A1
(43) Pub. Date: Jun. 1, 2017

(54) CONTROL DEVICE FOR A MEDICAL APPLIANCE

(71) Applicant: TRUMPF MEDIZIN SYSTEME GMBH + CO. KG, Saalfeld (DE)

(72) Inventors: Rudolf MARKA, Ismaning (DE); Deniz GÜVENC, Munich (DE); Stephan SCHRÖDER, Hannover (DE); Serhan ÖZHAN, Munich (DE); Andreas PÖSCH, Hannover (DE); Nina LOFTFIELD, Hannover (DE)

(21) Appl. No.: 15/322,343

(22) PCT Filed: Jun. 26, 2015
(86) PCT No.: PCT/EP2015/064549

§ 371 Date: Dec. 27, 2016

(30) Foreign Application Priority Data
Jun. 30, 2014 (DE) 10 2014 212 660.6

(51) Int. Cl. G06F 19/00; G05B 19/29; G06F 3/01; A61G 13/02; H05B 37/02; G05B 19/048; A61B 90/30

(52) U.S. Cl. CPC G06F 19/3406; G05B 19/048; G05B 19/29; A61B 90/30; A61G 13/02; H05B 37/0227; G06F 3/017; G05B 2219/32287; G05B 2219/50151; A61G 2203/12; A61G 2203/36

(57) ABSTRACT

A control device (2), for a medical apparatus, having at least one sensor (5) for three dimensionally detecting an object (8) which is configured to render graspable a vector (10) for a direction by its alignment is provided, wherein the object (8) is directed to a target area, and the vector (10) generates an intersection point with a surface of the target area. The control device (2) is adapted to recognize in which target area the intersection point is located and to actuate a predefined action for the target area by the medical apparatus.

The invention relates to a control device for a medical apparatus, in particular to a control device by which a contactless operation of the medical apparatus is possible.

Medical apparatuses, e.g. surgical tables or surgical lamps which are operated at the apparatuses themselves, at operation panels at the wall, or at remote controls, are known. Thereto, it is necessary that the operator is in the vicinity of theses operating elements. Furthermore, a surgeon himself cannot operate the apparatuses since the operating elements usually are located outside of the sterile area and the surgeon would become nonsterile by touching the operating elements.

Therefore an increased coordination effort is necessary to operate the apparatuses. Firstly, the operator has to be addressed by the surgeon, he has to come near the operating elements or to find the remote control, as the case may be, to check the orientation of the operating elements with respect to the apparatus, to start and stop, and, as the case may be, to correct the action.

The invention has been made based on the object to remedy the above disadvantages and to provide a control device for medical apparatuses allowing a contactless, simple operation of the medical apparatuses so that the surgeon can operate the apparatuses himself without becoming nonsterile.

The object is achieved by a control device according to claim 1 and a method for controlling medical apparatuses according to claim 15.

By the control device according to the invention, it is possible to detect an object, e.g. a finger of a surgeon, and its direction. By means of a sensor, it is possible to detect to which element to be activated of a medical apparatus, the object is directed, wherein a predefined action of the element is executed. Thereby, a contactless operation of the medical apparatus is possible and an intuitive operation of the medical apparatus is also enabled.

Now, the invention is elucidated by means of embodiments referring to the attached drawings.

In particular

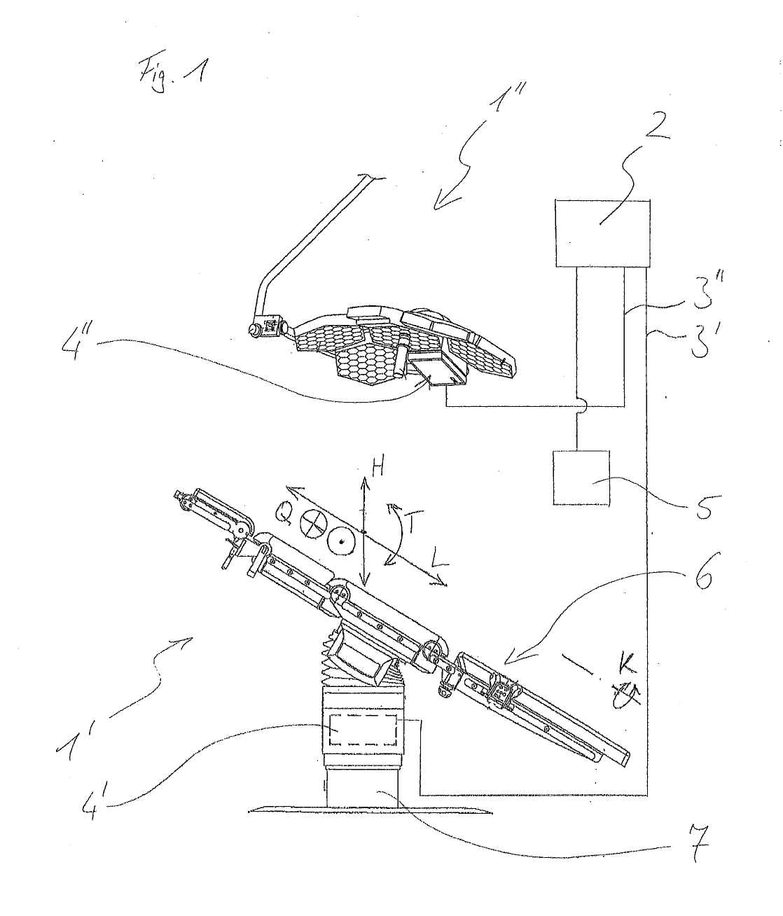

FIG. 1 shows a surgical lamp and a surgical table as two examples for medical apparatuses with a 3D sensor and a control device according to the invention,

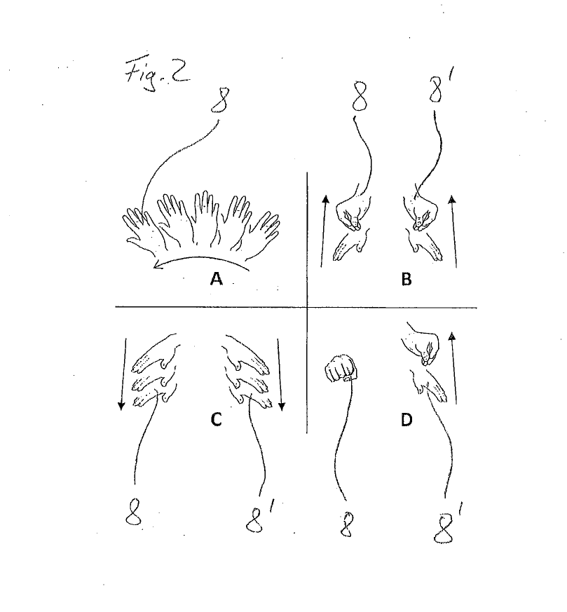

FIG. 2 shows different motions of hands for controlling a medical apparatus,

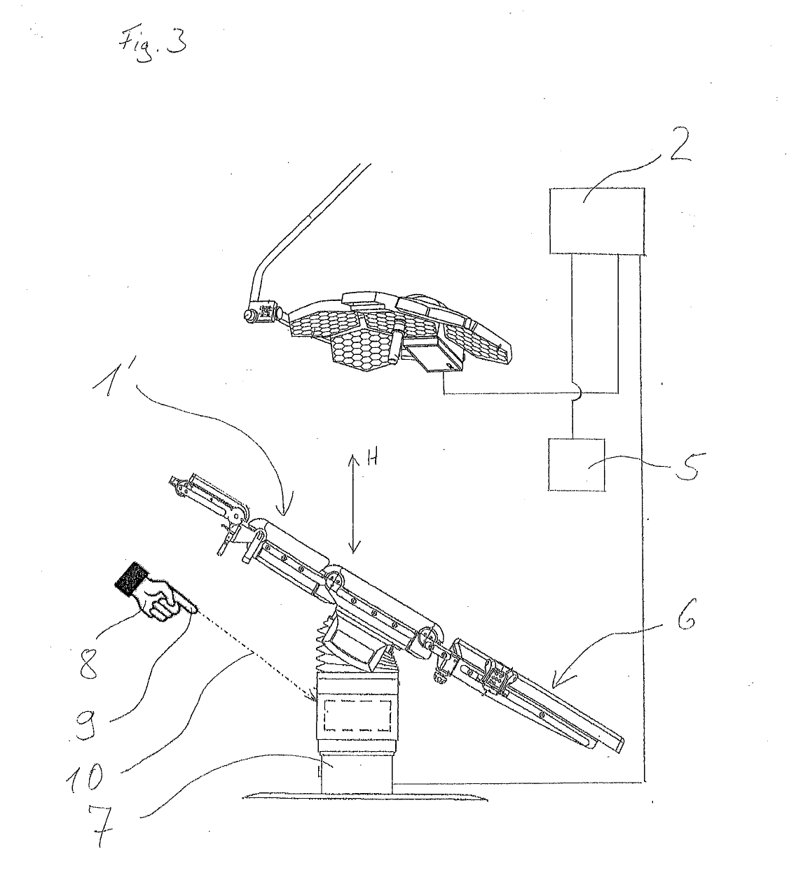

FIG. 3 shows an utilization of the control device, and

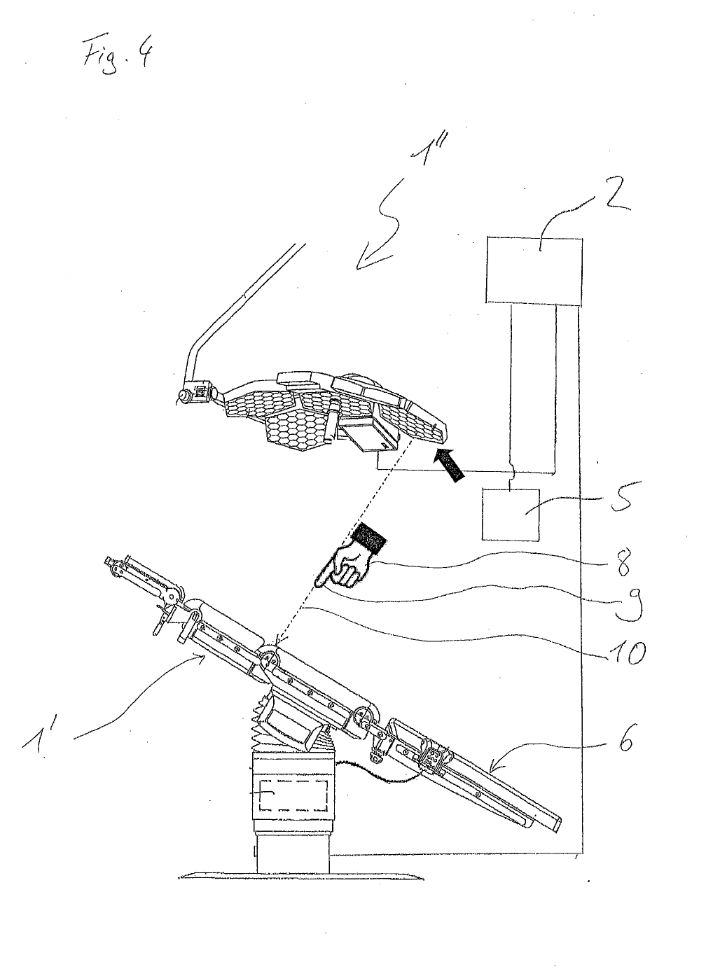

FIG. 4 shows further utilizations of the control device.

In FIG. 1, a surgical table 1′ and a surgical lamp 1″ are shown as examples for medical apparatuses to be controlled. The surgical table 1′ and the surgical lamp 1″ are connected to a control device 2 via data lines 3′. Particularly, a controller 4′ of the surgical table 1′ and a controller 4″ of the surgical lamp 1″ are connected to the control device 2 via the data lines 3′, 3″. Here, the connection is established via the data lines 3′, 3″; however, it can also alternatively be established in a wireless manner via radio or infrared. The control device 2 can also alternatively be included in the controller 4′ of the surgical table 1′ or in the controller 4″ of the surgical lamp 1″, therefore, principally, in the controller of a medical apparatus.

Furthermore, a 3D sensor 5 is connected to the control device 2. By the 3D sensor 5, objects in the room, their shape, position and motion are detected. Here, by detecting the motion is meant that a translation or rotation of the entire object in a certain motion sequence, a deformation by a motion of certain portions of the object, as well as a combination of the translation or rotation and the deformation of the object are detected. In an alternative embodiment, several 3D sensors are provided to detect the objects from different directions. Thus an enhanced reliability upon detection of the object and a larger space where the object can be detected is possible. Optionally, the 3D sensor is or the 3D sensors are fixed to a room ceiling so that the employment is less critical in view of deteriorations. Alternatively, the at least one 3D sensor 5 is attached to or integrated in the surgical table 1′ or the surgical lamp 1″, therefore, in the medical apparatus itself. The Utilization of a 3D sensor represents a suitable sensor selection, wherein the utilization is not limited to a 3D sensor but another suitable sensor can be employed. The gesture intended for the control can also be recognized by the utilization of camera systems for image recognition.

In one embodiment, the 3D sensor 5 is a so-called ToF (Time of Flight) camera. Here, the distances from the camera are measured for several pixels in that way that the object is illuminated by a light pulse, wherein the light is reflected by the object. The time needed by the light from emitting until the return to the ToF camera is measured for each pixel, wherein the time is proportional to the distance. Thereby, the object is not scanned but the entire object is simultaneously recorded.

The control device 2 is adapted to transform the motions of the objects into predefined actions of the medical devices.

The actions of the surgical lamp 1″ are e.g. a brightness change, a change of the light field diameter, a change of a focus situation, a change of a color temperature of the emitted light, or a change of the active zones where illuminants of the surgical lamp 1″ are activated.

The surgical table 1′ comprises a patient support plate 6 and a column 7. In the patient support plate 6 and in the column 7, several drives by which the patient support plate 6 can be travelled are provided.

The actions of the surgical table 1′ are e.g. a longitudinal displacement L whereupon the patient support plate 7 is displaced along its longitudinal axis, or a transversal displacement Q, whereupon the patient support plate 7 is displaced transversely with respect to its longitudinal axis. Furthermore, a swing K, whereupon the patient support plate 7 is pivoted around its longitudinal axis, and a Trendelenburg adjustment or an Antitrendelenburg adjustment, whereupon the patient support plate 7 is pivoted around its transversal axis are possible. Moreover, there is the option to change the height of the entire patient support plate 7 by a height adjustment H. Finally, individual segments of the patient support plate 7 can be pivoted with respect to one another to carry out specific supports for certain procedures at a patient.

The selection of the medical apparatus to be controlled amongst several medical apparatuses, therefore, here, whether an action of the surgical table 1′ or of the surgical lamp 1″ is to be triggered, is done by activation of the medical apparatus via a physical or virtual operating unit by the execution of a gesture in a detection space assigned to the medical apparatus or one of its components or to functions by the use of medical device-depending gestures and/or by a gesture-based selection as specified below.

Basically, depending on the medical apparatus and/or the selected component to be controlled, identical gestures can be transformed into various control instructions. The detection space assigned to a medical apparatus, components and/or functions can also only be relevant for a login or logout gesture, whereas the gesture for the control instruction itself can be again outside of this space if this seems advantageous. However, upon defined detection spaces, a login/logout gesture can be waived if the detection spaces is usually not used without intention to control.

FIG. 2 exemplarily shows four gestures, therefore, motions of hands as objects, the motions of which are transformed, by the control device 2, into a control instruction for a certain action of the surgical table 1′ as example for the medical device.

Illustration A shows a waving by one single hand 8. Here, the hand is held flat and it is not deformed, therefore, e.g. not clenched, during the waving, thus, during the translation or rotation.

Illustration B shows a motion of two hands 8, 8′, wherein the hands 8, 8′ execute a deformation, namely, starting from a flat hand into a shape in which, based on a gripping, the tips of the fingers are joined, and a translation, namely top-down. The motion principle can also be directly transferred to a one-hand gesture.

In illustration C, a motion in which both hands 8, 8′ are maintained flat and are moved top-down is shown.

In illustration D, a gesture in which one of the hands 8 clenched to a first remains at one place, whereas the other hand 8′ is deformed as described to illustration B and is then moved bottom-up or in a bow around the first of the other hand.

The control device 2 is adapted to process a specific one of the gestures as login motion and as logout motion. Here, illustration A shows the login motion as an authorization gesture. Only after the login motion has been executed, the control device 2 is adapted to process further motions of the hand or of the hands such that they are transformed into control instructions for the surgical table 1′.

After an execution of the intended motion, the logout motion is executed in turn, wherein the control device 2 is adapted such that this motion is then understood as logout motion so that no further motions are, by the control device, transformed into a control instruction for an action of the surgical table 1′. Merely an anew login motion is processed in turn. Alternatively, the login motion and the logout motion can also be different.

In the illustration B, the gesture by which the height adjustment H is activated and the patient support plate 7 is upwardly moved in this embodiment of the surgical table 1′ is illustrated. The adjustment is possible for the entire table; however, it can also be provided only for selected segments, e.g. by an execution of a gesture in a detection space assigned to the segment. For the height adjustment, the gesture is executed by several hands 8, 8′ executing a predefined motion, namely, the deformation, in particular, starting from a flat hand into a shape in which the tips of the fingers are joined, and executing a subsequent bottom-up translation of the hands. The control device 2 is adapted to recognize whether several hands 8, 8′ execute the predefined motion and transforms the detected motions according to a combination of the detected motions into the predefined action, here the upward motion of the patient support plate 7. A one-hand gesture with identical or alternative gesture shape is also conceivable.

Also in the illustrations C and D, the gesture is executed by several hands 8, 8′, wherein in illustration C, both flat hands are downwardly moved in a mere translation so that the height adjustment H is activated and the patient support plate 7 is downwardly adjusted. In illustration D, the one hand is however not moved in a translational manner or deformed but merely the shape, therefore the hand 8 as a fist, is detected. The other hand 8′ executes a translation and a deformation of the hand. Thereby, a Trendelenburg adjustment or an Antitrendelenburg adjustment are actuated.

The motion as well as the deformation of the hand 8, 8′ are then detected in that case if they are executed at a certain speed within a predetermined tolerance range. If a motion is executed too fast or too slow, it is not detected as a predefined motion but it is ignored.

The speed of a motion instruction can basically correspond to the speed of the gesture motion or it can be in a defined relationship, it can be related to a distance of the object or to a plane in the detection space and/or it can depend on an additional gesture.

The predefined actions of the surgical table 1′ are optionally executed in real time which means that the control device 2 is adapted such that the detected motions are immediately executed by the surgical table 1′.

Besides the direct control of the medical apparatus, the user can alternatively control it via a model. The model of the medical apparatus is illustrated on a user interface or a reproducing unit, such as a monitor. If the model is activated by motion instructions, the medical apparatus itself analogously moves. Inasmuch as not a motion but another action shall be initiated, the position and/or the respective motion or activation instruction is comprehensible at the reproducing unit via a cursor, as the case may be, in different appearances depending on the instruction. Hereby, the safe operation increases by the visual comprehensibility of the action of the user. Moreover, the operation can be spatially separated from the medical apparatus to be operated while the visual surveillance of the instructions is maintained.

In alternative embodiments, the assignment of the gestures and the predefined actions of the medical device distinguishes from this embodiment. Furthermore, there is the option to additionally or alternatively detect also other gestures or gestures of other objects, e.g. of legs.

In FIG. 3, an alternative or a supplement of a control of the surgical table 1′ is shown. Also here, a motion of the hand 8 is detected by the 3D sensor 5 or by the other sensor and forwarded to the control device 2. In particular, the motion is detected in combination with an alignment of an extended finger 9. In context with the extended finger 9, the hand is to be regarded as rod-like. This means that it is possible to detect the alignment of the hand 8 with the extended finger 9. The alignment is detected as an axis of the finger by detected points of the finger 9. A vector 10 is defined as an extension of the connection of these points. Alternatively, other points of the hand 8 and of the finger 9 are used for the definition of the vector 10. It is essential that an unambiguous vector definition is possible via the points. In further alternatives, the vector 10 is determined from the orientation of another rod-like object which is, for example, held in the hand 8.

In this embodiment, the 3D sensor 5 detects several elements of the surgical table 1′ which are controllable, therefore to be activated. Here, the element is exemplarily the drive in the column 7 for the height adjustment. The control device 2 recognizes from the direction of the vector 10 and a position of the hand 8, thus from the vector 10 starting from the hand 8, that there is an intersection point of the vector 10 and the column 7 and recognizes the element to be activated that has to execute a predefined action by the position of the intersection point. As described above, the predefined action is then executed by detected predefined motions and/or the gestures and/or the detection space of the hand 8 or of the hands 8, 8′ assigned to a functionality/a control instruction. In an alternative embodiment, the control device 2 is adapted such that the predefined action is executed by the detection of an instruction being another than a gesture, for example by a voice input.

In FIG. 4, a further option of the control, here, by means of the surgical lamp 1″, is shown.

The control device 2 is here configured such that the vector 10 is in turn determined via the points on the finger 9 and via its axis. The control device 2 recognizes the direction of the vector 10 via the detection of the points by the 3D sensor 5. Furthermore, the control device 2 detects, by the 3D sensor, human bodies being located in its detection space.

Also here, an intersection point of the vector 10 and the human body (not shown) is detected. The control device 2 is adapted such that it executes an action selected in advance with respect to the patch of the human body. In FIG. 4, it is shown that the vector is directed to a patch on the patient support plate 6. The action selected in advance, selected e.g. by the pointing to the surgical lamp 1′, is then executed such that the patch on the patient support plate 6 on which the human is lying is illuminated. This happens e.g. by a motorized adjustment of modules of the surgical lamp 1″ or by activating illuminants directed to the patch.

In FIG. 4, furthermore, a function of an optional assimilation of the control device 2 is shown. Additionally to the intersection point with the human body, the spatial orientation of the vector 10 also determines, in the direction of the vector, a second intersection point with a element of the medical apparatus to be activated in the opposite direction of the vector. The action selected in advance, here, the illumination of the patch on the patient support plate 6, is then executed in the direction of the spatial orientation of the vector 10 by the object on which the intersection point of the vector 10 with the object is located. Here, it is shown that the patch on the patient support face 6 is illuminated by the module of the surgical lamp 1″ identified by an arrow.

The use of the vector 10 can further be utilized to generate an intersection point with a surface of a target area, e.g. the patient support of the surgical table or the patient being located thereon. If this intersection point can be generated, at the first set-out, the indication of direction to a functionality to be joined therewith is reasonable and it is executed. If the plausibility check fails since a selection e.g. come to nothing, the control instruction is not executed or it has to be explicitly confirmed by the user.

The functions assigned to the surgical table 1′ and the surgical lamp 1″ can analogously be executed by all suitable medical apparatuses connected to the control device 2. The different embodiments can be combined with one another.

In use, the control device 2 firstly detects, by the 3D sensor, whether the predefined login motion is executed.

Subsequently to the predefined login motion, the motion of the object, therefore, of the hand 8, 8′ or of the arm is detected by the 3D sensor and the predefined action of the medical apparatus, assigned to the predefined motion of the object, is actuated by the control device 2.

After a completion of the requested predefined action of the medical apparatus, the logout motion is executed for terminating the operation so that, after the logout motion, no further motions are interpreted as actions to be executed.

Optionally, not only the motion of one of the objects but of several objects are detected. Here, the position and the motion of both hands 8, 8′ are detected and the predefined action is executed according to the combination of the motions of the several objects.

Upon the optional function in which it is possible to the select the element of the medical apparatus to be activated, after the activation and before the execution of the motion for activating, the requested element to be activated is selected by pointing to this element by the finger 9 (directing the rod-like object). Then, the predefined motion is executed by the objects and the control device 2 actuates the requested element to be activated so that the action according to the predefined motion is executed.

Upon the further optional function in which the patch to which the action of the element of the medical apparatus is directed is selected, it is pointed to the patch relating to which the action shall be executed by the finger 9 after an optionally necessary activating of the element. Thereby, the control device 2 activates the requested element such that the action relating to the patch is executed, therefore, the patch on the patient support face 6 is illuminated.

Upon the further optional function that, by the direction from where it is pointed, also the element that shall execute the action is activated, the direction of the finger 9 (of the rod-like object) is detected and the element to be activated opposite with respect to the direction of the finger which shall execute the action is selected additionally to the patch where the action is to be executed. The element to be activated is then actuated so that the action is executed from the direction of the finger 9.

What is claimed is:

1.-19. (canceled)

20. A control device, for a medical apparatus, having at least one sensor for three-dimensionally detecting an object, wherein the object is configured to render graspable its position and, by its alignment, a vector for a direction, wherein
the object is directed to a target area, and
the vector starting from the object generates an intersection point with a surface of the target area,
the control device is adapted to recognize in which target area the intersection point is located and to actuate an action predefined for the target area by the medical apparatus.

21. The control device of claim 20, wherein
the control device is adapted to execute the predefined action relating to the target area only if a plausibility check of the intersection point is passed, or to execute the predefined action due to the vector defined by the object and its direction if an additional confirmation is provided.

22. The control device of claim 20, wherein
the object is configured rod-like or comprises at least a rod-like portion comprising a free end, and the control device is adapted to determine a direction by the free end and an axis of the rod-like object.

23. The control device of claim 20, wherein
the at least one sensor is configured to recognize a human body, and
the control device is adapted to recognize to which patch of the human body the object is directed, and to actuate the predefined action relating to the patch of the human body.

24. The control device of claim 20, wherein
the at least one sensor is configured to recognize elements to be activated of the medical apparatus, and
the control device is adapted to recognize, to which elements to be activated the object is directed, and to induce the predefined action related to the recognized elements to be activated.

25. The control device of claim 20, wherein
the control device is adapted to determine a direction of the predefined action from a spatial orientation of the object and its direction, and to actuate the medical apparatus such that the predefined action is executed by the element to be activated being located opposite to the direction.

26. The control device of claim 20, wherein
the at least one sensor is additionally configured to detect a motion of the object, and
the control device is adapted
to transform a predefined motion of the at least one object into a control instruction for the predefined action of the medical apparatus.

27. The control device of claim 20, wherein the at least one sensor is a ToF camera adapted to detect the object by a running time of emitted and reflected light.

28. The control device of claim 20, wherein the control device is adapted
to process a predefined login motion of the object such that the further predefined motions of the object are transformed into the control instruction for the predefined action of the medical apparatus, and
to process a predefined logout motion of the object such that no further motions of the object are transformed into a control instruction for an action of the medical apparatus.

29. The control device of claim 28, wherein the control device is adapted to recognize whether several objects execute a respectively predefined motion, and to transform the predetermined control instruction for the predefined action of the medical apparatus according to a combination of the respective motion of the several objects.

30. The control device of claim 28, wherein the control device is adapted to execute the predefined action in real time.

31. The control device of claim 28, wherein several sensors are provided.

32. The control device of claim 28, wherein the medical apparatus comprises a surgical table.

33. The control device of claim 28, wherein the medical apparatus comprises a surgical lamp.

34. The control device of claim 33, wherein
the predefined action is a change of a focus situation so that a focus point of light beams of the surgical lamp is located on the surface of the target area.

35. A method for controlling a medical apparatus with a control device comprises the following steps:
detecting a vector and a surface of target areas by the at least one sensor,
determining an intersection point of the vector and the target area, to which an object is directed, and
actuating a predefined action of the medical apparatus associated with the target area.

36. The method for controlling a medical apparatus of claim 35 further comprising:
detecting, with the sensor, elements of the medical apparatus to be activated,
detecting, with the sensor, to which elements of the medical apparatus the object is directed, and
actuating the predefined action of the element to be activated to which the object is directed.

37. The method of claim 35, wherein the control device executes a predefined action relating to the target area only if a plausibility check of the intersection point is passed, or executes a predefined action due to the vector determined by the object and its direction if an additional confirmation is provided.

38. The method of claim 35, further comprising:
detecting a direction of the object from its orientation and its end,
detecting the element of the medical apparatus to be activated which is located opposite to the direction of the element, and
actuating the element to be activated executing the predetermined action from the direction of the object.
